(19) United States
(12) Patent Application Publication
(10) Pub. No.: US 2013/0056419 A1
(43) Pub. Date: Mar. 7, 2013

(54) DIALYSATE MIXING AND DIALYZER CONTROL FOR DIALYSIS SYSTEM

(71) Applicant: James R. Curtis, Sunnyvale, CA (US)

(72) Inventor: James R. Curtis, Sunnyvale, CA (US)

(21) Appl. No.: 13/598,176

(22) Filed: Aug. 29, 2012

Related U.S. Application Data
(60) Provisional application No. 61/529,157, filed on Aug. 30, 2011.

(51) Int. Cl. A61M 1/16

(52) U.S. Cl. 210/647; 210/101; 210/90

(57) ABSTRACT

A medical dialysis system including a filtration system configured to filter a water stream, a water purification system configured to purify the water stream in a non-batch process, a mixing system, and a dialyzer system. The mixing system includes a supply of dialysate components, a conductivity sensor positioned within a fluid pathway through which the water stream flows, and a control mechanism having a pump configured to control an amount of the one or more dialysate components added to the water stream from the supply. The mixing system can produce a stream of dialysate from mixing the one or more dialysate components with the water stream in a non-batch process. The dialyzer system includes a dialyzer, a plurality of pumps capable of pumping the stream of dialysate across the dialyzer, and another conductivity sensor positioned downstream of the dialyzer within a fluid pathway through which the water stream flows.

### REFERENCE TO RELATED APPLICATIONS

This application claims priority of U.S. Provisional Patent Application Ser. No. 61/529,157, filed on Aug. 30, 2011 and entitled DIALYSATE MIXING AND DIALYZER CONTROL FOR DIALYSIS SYSTEM. The disclosures of the Provisional patent application is hereby incorporated by reference in its entirety.

This application is related to the following U.S. patent applications: (1) U.S. patent application Ser. No. 12/795,371, entitled “Microfluidic Devices” and filed Jun. 7, 2010, which claims priority to U.S. Provisional Patent Application Ser. No. 61/220,117, filed on Jun. 24, 2009; (2) U.S. patent application Ser. No. 12/795,498, entitled “Dialysis System With Ultrafiltration Control” and filed Jun. 7, 2010, which claims priority to U.S. Provisional Patent Application Ser. No. 61/267,043, filed on Dec. 5, 2009; (3) U.S. patent application Ser. No. 12/795,382, entitled “Fluid Purification System” and filed Jun. 7, 2010; and (4) U.S. patent application Ser. No. 12/795,444, entitled “Dialysis System,” filed Jun. 7, 2010, which claims the benefit of priority of U.S. provisional application Ser. No. 61/220, 117, filed Jun. 24, 2009 and to provisional application Ser. No. 61/267,043, filed Dec. 5, 2009. This application is also related to International Patent Application No. PCT/US2010/037621, entitled “Microfluidic Devices,” and filed Jun. 7, 2010. The disclosures of the aforementioned patent applications are hereby incorporated by reference in their entirety.

### BACKGROUND

The present disclosure concerns a dialysis system, such as a microfluidic or flow field dialyzer capable of being fluidly coupled to a dialysate stream and a blood stream, and a method for using the dialysis system.

There are, at present, hundreds of thousands of patients in the United States with end-stage renal disease. Most of those require dialysis to survive. United States Renal Data System projects the number of patients in the U.S. on dialysis will climb past 600,000 by 2012. Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Given the demanding nature of in-center dialysis, many patients have turned to home dialysis as an option. Home dialysis provides the patient with scheduling flexibility as it permits the patient to choose treatment times to fit other activities, such as going to work or caring for a family member. Unfortunately, current dialysis systems are generally unsuitable for use in a patient's home. One reason for this is that current systems are too large and bulky to fit within a typical home. Current dialysis systems are also energy-inefficient in that they use large amounts of energy to heat large amounts of water for proper use. Although some home dialysis systems are available, they generally use complex flow-balancing technology that is relatively expensive to manufacture and most systems are designed with a system of solenoid valves that create high noise levels. As a result, most dialysis treatments are performed at dialysis centers.

### SUMMARY

In view of the foregoing, there is a need for improved aspects of preparing and purifying fluids for, and controlling sessions of, dialysis to ease the burden on the patient in a home-dialysis setting, to make the treatment more efficient, and/or to permit the patient more flexibility when receiving dialysis to match aspects of his or her lifestyle. Such aspects include improved devices for heating, purifying and cooling in-line water streams for use in preparing dialysate, the use of conductivity to substantially match various levels of one or more components (such as sodium) in dialysate to the levels of such components in a patient's blood; use of a pressure transducer downstream of the components of dialysate make-up as indicator to the patient of whether bag has remaining sufficient fluid to continue treatment; effecting control of a dialyzer to accommodate shorter treatment periods; and the ability of the patient or system to control between low and high flow states to ensure all dialysate is used even for shorter treatment periods.

In one aspect, disclosed is a medical dialysis system including a filtration system configured to filter a water stream; a water purification system configured to purify the water stream in a non-batch process; a mixing system, and a dialyzer system. The mixing system includes a supply of one or more dialysate components; a first conductivity sensor positioned within a fluid pathway through which the water stream flows; and a control mechanism having a pump configured to control an amount of the one or more dialysate components added to the water stream from the supply. The mixing system is configured to produce a stream of dialysate from mixing the one or more dialysate components with the water stream in a non-batch process. The dialyzer system includes a dialyzer, a plurality of pumps capable of pumping the stream of dialysate across the dialyzer, and a second conductivity sensor positioned downstream of the dialyzer within a fluid pathway through which the water stream flows. The dialyzer is configured to fluidly couple to the stream of dialysate and a blood stream and having a membrane separating the stream of dialysate from the blood stream facilitating dialysis of the blood stream.

The system can further include a microprocessor configured to calculate a difference between a first electrical measurement by the first conductivity sensor and a second electrical measurement by the second conductivity sensor to determine a transfer rate of electrolytes between the stream of dialysate and the blood stream within the dialyzer. The control mechanism of the mixing system can adjust in real-time the amount of the one or more dialysate components mixed into the water stream based on the calculated difference until the transfer rate of electrolytes falls below a predetermined threshold. The transfer rate of electrolytes falls below a predetermined threshold as the difference between the first electrical measurement and the second electrical measurement approaches zero. The electrolyte level of the blood stream can remain unchanged following a treatment with the dialysis system. The mixing system can further include a pressure transducer positioned downstream of the supply and upstream of the control mechanism. The pressure transducer can measure fluid pressure from the supply and indicate whether the supply contains sufficient components to continue a dialysis treatment based on the measured fluid pressure. The control mechanism can monitor the measured fluid pressure from the pressure transducer and compare the measured fluid pressure to a threshold value to assess a status of the supply. The control system can provide an alert regarding the status of the supply. The alert can indicate a degree of sufficiency or insufficiency of the supply.

In another aspect, disclosed is a method for matching dialysate electrolyte levels to a patient's blood electrolyte levels during a dialysis treatment. The method includes purifying a water stream in a non-batch process using a water purification system; producing a dialysate stream by mixing one or more dialysate components with the water stream in a non-batch process; measuring electrical conductivity of the dialysate stream using a first conductivity sensor positioned within a fluid pathway upstream of a dialyzer before the dialysate stream contacts a blood stream, the dialysate stream having a first electrolyte concentration; measuring electrical conductivity of the dialysate stream using a second conductivity sensor positioned within a fluid pathway downstream of the dialyzer after the dialysate stream contacts the blood stream, the dialysate stream having a second electrolyte concentration; calculating a difference between the first electrical conductivity measurement and the second electrical measurement to determine a transfer rate of electrolytes between the dialysate stream and the blood stream within the dialyzer at a first flow rate; and adjusting in real-time the first electrolyte concentration using a control mechanism comprising a pump configured to control an amount of the one or more dialysate components mixed with the water stream until the transfer rate of electrolytes falls below a predetermined threshold.

The transfer rate of electrolytes falls below a predetermined threshold as the difference between the first electrolyte concentration and the second electrolyte concentration approaches zero. The dialysate stream can contact the blood stream within the dialyzer across a semi-permeable membrane. The blood electrolyte levels can remain unchanged following the dialysis treatment.

Other features and advantages should be apparent from the following description of various embodiments, which illustrate, by way of example, the principles of the disclosed devices and methods.

### BRIEF DESCRIPTION OF THE DRAWINGS

FIG. 1 shows a high level, schematic view of an implementation of a dialysis system.

FIG. 2 shows a high level, schematic view of an exemplary implementation of a water purification system of the dialysis system.

FIG. 3 shows a schematic, plan view of an exemplary implementation of a microfluidic heat exchange system adapted to heat and cool a single fluid without the use of a second fluid stream to add heat to or remove heat from the fluid.

FIG. 4 shows a high level, schematic view of an exemplary implementation of a dialysate preparation system of the dialysis system.

FIG. 5 is a schematic, cross-sectional view of an exemplary implementation of a dialyzer of the dialysis system.

FIG. 6 shows a schematic view of an exemplary implementation of a flow balance system.

FIG. 7 shows a schematic view of another exemplary implementation of a flow balance system.

FIG. 8 shows a schematic representation of the flow balance system running in a calibration mode.

FIG. 9 shows a schematic representation of the flow balance system running in a dialysis mode.

FIG. 10 shows a schematic representation of a flow rate control mechanism in a flow path.

### DETAILED DESCRIPTION

Disclosed herein are small, lightweight, portable, systems that have the capability of reliably, reproducibly, highly efficiently and relatively inexpensively providing a source of purified water of sufficient volumes for home dialysis. In addition, the systems disclosed herein require much less purified water at any one time than the volumes typically needed for dialysis today, thereby further reducing the expense of running the system at home. In addition, the systems described herein are capable of producing real-time, on-demand ultrapure water for dialysis, the gold standard of present-day dialysis. Disclosed herein are in-line, non-batch water purification systems that use a microfluidics heat exchanger for heating, purifying and cooling water. The systems described herein consume relatively low amounts of energy. The systems described herein although suitable for use in a home dialysis system, can be used in other environments where water purification is desired. The systems can also be used to purify fluids other than water. As will be described in more detail below, the systems described herein can be connected to a residential source of water (such as a running water tap to provide a continuous or semi-continuous household stream of water) and can produce real-time pasteurized water for use in-home dialysis, without the need to heat and cool large, batched quantities of water.

Dialysis System

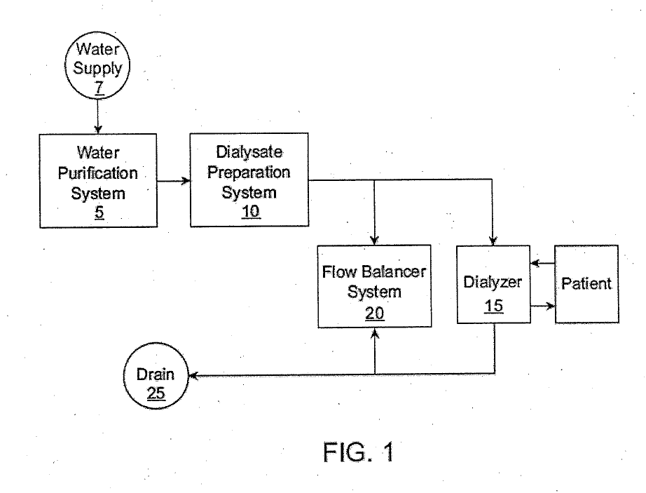

FIG. 1 shows a high-level, schematic view of an implementation of a dialysis system. The dialysis system can include a plurality of subsystems that collectively operate to receive and purify water, use the water to prepare dialysate, and supply the dialysate to a dialyzer that performs one or more various types of dialysis on the blood of a patient such as hemodialysis, ultrafiltration and hemodiafiltration. The dialysis system can include plumbing that provides fluid pathways for water, dialysis, and blood to flow through the dialysis system, as well as one or more pumps that interface with the plumbing for driving fluid flow through the system. The dialysis system can also include one or more sensors, such as fluid flow sensors, pressure sensors, conductivity sensors, etc. for sensing and reporting one or more characteristics of fluid flowing through the system.

In an embodiment, the entire dialysis system (including the water preparation and purification system, dialysate preparation system, flow balancer system, dialyzer, and hardware, such as plumbing and sensors) is contained within a single housing that is compact and portable. In addition, the dialysis system can prepare dialysate using a tap water, such as in a home or hotel room. In an embodiment, the entire dialysis system consumes less than about 22″ by 14″ by 9″ of space when dry, which generally corresponds to the size limit for carry-on baggage of an airline. In an embodiment, the entire dialysis system weighs less than about fifty pounds when dry.

With reference still to FIG. 1, the dialysis system can include a water preparation and purification system 5 that purifies water from a water supply 7. The water purification system 5 can supply the purified water to a dialysate preparation system 10 that uses the purified water to prepare dialysate. The dialysis system can further include a dialyzer 15 that receives the dialysate from the dialysate preparation system 10 and performs dialysis on a patient's blood. In an embodiment, the dialyzer 15 and the dialysate preparation system 10 both can interface with a flow balancer system 20 that regulates the flow of dialysate to the dialyzer to achieve different types of dialysis, including hemodialysis, ultrafiltration, and hemodiafiltration, as described in detail below.

Diffusion is the principal mechanism in which hemodialysis removes waste products such as urea, creatinine, phosphate and uric acid, among others, from the blood. A differential between the chemical composition of the dialysate and the chemical composition of the blood within the dialyzer causes the waste products to diffuse through a membrane from the blood into the dialysate. Ultrafiltration is a process in dialysis where fluid is caused to move across the membrane from the blood into the dialysate, typically for the purpose of removing excess fluid from the patient's blood stream. Along with water, some solutes are also drawn across the membrane via convection rather than diffusion. Ultrafiltration is a result of a pressure differential between a blood compartment and a dialysate compartment in the dialyzer where fluid moves from a higher pressure to a lower pressure. In some circumstances, by design or unintentional consequence, fluid pressure in the dialysate compartment is higher than the blood compartment causing fluid to move from the dialysate compartment into the blood compartment. This is commonly referred to as reverse ultrafiltration.

In hemodiafiltration, a high level of ultrafiltration is created, greater than the amount required to remove fluid from the patient's blood, for the purpose of increasing convective solute transport across the membrane. The amount of fluid in excess of what is required to be removed from the patient's blood must therefore be returned to the blood stream in order to avoid an adverse hemodynamic reaction. This is accomplished by intentionally periodically increasing the pressure in the dialysate compartment of the dialyzer to cause the appropriate amount of reverse ultrafiltration. This process of ultrafiltration alternating with reverse ultrafiltration is often referred to as “push-pull hemodiafiltration.” This is a significant improvement over more common methods of hemodiafiltration where sterile fluid is administered to the patient in a location outside of the dialyzer.

In use, the patient is coupled to the dialyzer 15 such that the patient's blood flows into and out of the dialyzer 15 using devices and techniques known to those skilled in the art. The dialysis system prepares dialysate using water from a household water source, such as a tap, that has been previously prepared through filtration and purification before being mixed with various dialysate components to make the dialysate, and then flows the dialysate through the dialyzer in communication with the blood such that one or more of the dialysis processes on the blood is performed. The water purification system includes a plurality of subsystems that collectively operate to purify the water including pasteurization of the water, as described more fully below. The purified water is then mixed with dialysate concentrates to form dialysate, which is supplied to the dialyzer 15 and to the flow balancer system, which regulates the flow of dialysate to the dialyzer 15 to selectively achieve different types of dialysis, including hemodialysis, ultrafiltration, and hemodiafiltration, as described more fully below. The dialysis system supplies the used dialysate to a drain 25. In an embodiment, the system recaptures heat from the used dialysate before going to the drain.

Subsystems of Dialysis System

Embodiments of the various subsystems of the dialysis system are now described, including the water purification system 5, dialysate preparation system 10, dialyzer 15, and flow balancer system 20. It should be appreciated that the descriptions are examples of implementations and that variations are possible.

A. Water Purification Sub-System

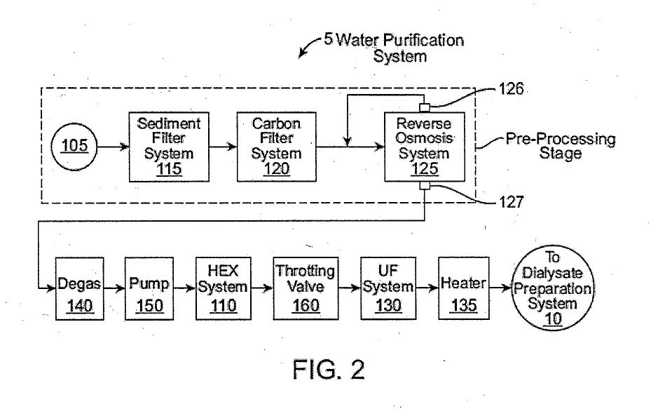

FIG. 2 shows a high level, schematic view of the water purification system 5. The water purification system 5 includes a plurality of subsystems and/or components each of which is schematically represented in FIG. 2. Although it is described in the context of purifying water, the water purification system 5 can be used to purify fluids other than water. Water enters the fluid purification system at an entry location 105 (from the water supply 7 in FIG. 1) and communicates with each of the subsystems and components as the water flows along a flow pathway toward the dialysate preparation system 10. The subsystems may include, for example, a sediment filter system 115, a carbon filter system 120, a reverse osmosis system 125, an ultrafilter system 130, an auxiliary heater system 135, a degassifier system 140, or any combination thereof.

Upon exiting the fluid purification system 5, and prior to entering the dialysate preparation system 10, the fluid is in a purified state. This preferably includes the fluid being in a pasteurized state although the fluid system does not necessarily pasteurize the fluid in all circumstances. The embodiment shown in FIG. 2 is exemplary and not all of the components shown in FIG. 2 are necessarily included in the water purification system 5. The individual components included in the system may vary depending on the type and level of purification or pasteurization required. The quantity and sequential order of the subsystems along the flow pathway shown in FIG. 2 is for purposes of example and it should be appreciated that variations are possible.

A method for purifying water using the fluid purification system 5 is now described including a description of a fluid flow path through the system. As mentioned, water can enter the water purification system 5 via an entry location 105. The entry location may include a three-way valve that may be set such that incoming water is received from one of at least two water sources. One such water source may be household water tap. Alternately, the valve may be set to receive recirculated water that was previously routed through the water purification system 5 and that is re-routed back into the system such as to flush the system. When the valve is set to receive recirculated water, the re-circulated water may bypass one or more of the subsystems as it flows through the water purification system 5.

When the valve is set to receive water from the household water tap, the incoming water can first flow through at least one sediment filter system 115, which includes one or more sediment filters that filter sediment from the water flowing therethrough. In an embodiment, the sediment filter 115 removes particulate matter down to 5 microns or even 1 micron. A pressure sensor may be positioned upstream of the sediment filter(s) and a pressure sensor may also be positioned downstream of the sediment filter(s) in order to monitor flow conditions. In addition, the flow pathway may include one or more pressure regulators configured to regulate fluid pressure to achieve a desired flow rate through the system. The pressure regulator(s) may be used to compensate for a household tap having a flow rate that is above or below a desired range.

The water can then flow through a carbon filter system 120, which can include one or more carbon filters that filter materials such as organic chemicals, chlorine and chloramines from the water. In an embodiment, the carbon filter system 120 includes two carbon filters with a sample port positioned in the flow path between the carbon filters. The sample port provides an operator with access to the water flowing through the system, such as for quality control purposes. In an embodiment, at least one pressure sensor and at least one conductivity sensor are positioned in the flow pathway downstream of the carbon filter system 120. The conductivity sensor provides an indication as to the percentage of dissolved solids contained in the water. In addition, one or more pumps may be positioned at various locations along the water flow pathway such as between the filter subsystems.

The water can flow from the carbon filter system 120 to a reverse osmosis system 125 configured to remove particles from the water pursuant a reverse osmosis procedure. The reverse osmosis system 125 can remove greater than 95% of the total dissolved solids from the water. The reverse osmosis system 125 may have two outlets including a waste water outlet 126 and a pure water outlet 127. The waste water outlet 126 outputs waste water from the reverse osmosis system 125. The waste water can be rerouted back into an upstream location of the water pathway for re-entry into the reverse osmosis system 125. In this regard, a sensor such as a conductivity sensor may be located upstream of the reverse osmosis system 125 as a means of verifying the contents of the water. Alternately, the waste water outlet 126 may supply the waste water to a drain.

The sediment filter system 115, carbon filter system 120, and reverse osmosis system 125 collectively form a pre-processing stage that removes a majority of dissolved solids, bacteria contamination, and chemical contamination, if any, from the water. The water is therefore in a somewhat macro-purified state as it exits the pre-processing stage. Thus, the preprocessing stage supplies relatively clean water to the downstream pump(s) and also to a downstream heat exchange system 110 that pasteurizes the water. The preprocessing stage can reduce or eliminate the potential for scale build-up and corrosion during heating of the water by the heat exchange system 110.

One or more degassifier systems 140 may be positioned in the flow pathway upstream and/or downstream of the heat exchange system 110 for removing entrained gas from the water. The degassifier system 140 may include any of a variety of components adapted to remove entrained gas from the water. For example, the degassifier systems 140 may include a degassing membrane, a spray chamber and/or a bubble trap.

After the water passes the pre-processing stage, the water can flow through a pump 150 that pumps the water into the heat exchange (HEX) system 110. The heat exchange system 110 can heat the water to a temperature that achieves pasteurization of the water. In an embodiment, the heat exchange system 110 is a microfluidic heat exchange system. Embodiments of microfluidic heat exchange systems are described in detail in U.S. patent application Ser. No. 12/795,382, filed Jun. 7, 2010 and U.S. Patent Application Publication No. 2010/0326916, filed Jun. 7, 2010, which are each incorporated by reference in their entireties.

The pump 150 may be used to increase the water pressure to a level higher than the saturation pressure encountered in the heat exchange system 110. This prevents phase change of the water inside the heat exchange system 110. Thus, if the highest temperature reached in the heat exchange system 110 is 150 degrees Celsius where the water would have a saturation pressure known to one of skill in the art, the pressure of the water coming out of the pump would exceed that saturation pressure by a certain safety margin, such as 10 psi, to ensure that no phase change occurs. The pump can increase the water pressure to a level that is at or exceeds the saturation pressure to ensure no localized boiling. This can be important where the heat exchange system is used to pasteurize water and the water is exposed to high temperatures that may be greater than 138 degrees Celsius, i.e., well above the boiling point of water at atmospheric pressure.

After leaving the heat exchange system 110, the water can pass into a throttling valve 160, such as flow restrictor, which maintains the pressure though the water path from the pump 150 to outlet of the heat exchange system 110. A pressure transducer can be positioned in the line after the heat exchange system 110, and before entering the throttling valve 160 to control the pump 150. The throttling valve 160, which are typically positioned downstream from the heat exchange system 110, can control the flow rate. The throttling valve 160 and the pump 150 may be controlled and adjusted to achieve a flow rate and a desired pressure configuration. The pump 150 and the throttling valve 160 may communicate with one another in a closed loop system to ensure the required pressure is maintained for the desired flow rate and temperature. One or more temperature sensors and/or flow sensors may be positioned along the flow pathway downstream of the heat exchange system for use in controlling the pump 150 and the throttling valve 160.

After the water leaves the throttling valve 160, it can pass to an ultrafilter (UF) system 130 that removes macromolecules and all or substantially all of the dead bacteria killed by the pasteurization process from the water to ensure no endotoxins remain in the water before mixing the dialysate. The presence of macromolecules may be detrimental to the dialysis process. The water can then pass through a heater system 135 that may, if necessary or desired, heat the water to a desired temperature, such as to normal body temperature (98.6 degrees Fahrenheit). From the heater system 135, the water can pass to the dialysate preparation system 10.

In an embodiment, a second heat exchange system is positioned in the flow pathway upstream of the heater system 135. The second heat exchange system can be used to further cool the water that comes out of the heat exchange system 110 in the event that the water is above a predetermined desired temperature, such as 37 degrees Celsius. The second heat exchange system may be connected to a separate source of cool water that will then act as a cooling agent or it can be connected to the water rejected from the reverse osmosis system 125. The second heat exchange system may be used in environments where the water source produces very warm water and/or when the heat exchange system 110 is unable to cool the water sufficiently for use in dialysis.

In an embodiment, the dialysis system can include a control system that allows a user to set or adjust the temperature of the dialysate prior to the dialysate entering the dialyzer. For example, the user can interact with the control system to raise or lower the temperature of the dialysate to accommodate for ambient temperature, such as on a particularly hot day or a particularly cold day.

Microfluidic Heat Exchanger

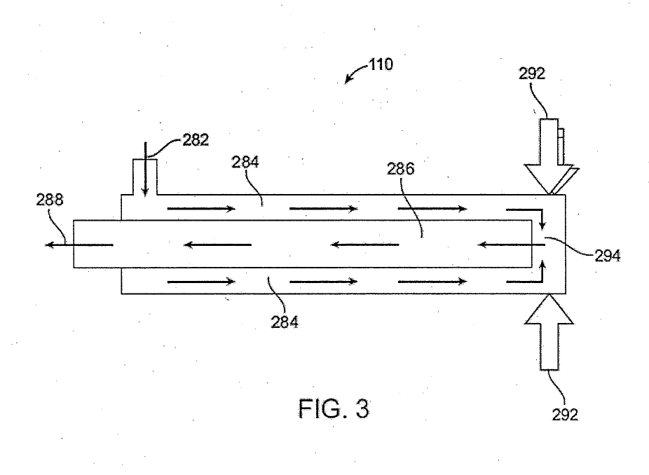

As discussed above, the water purification system 5 may employ a heat exchange system 110 that is adapted to pasteurize the water. FIG. 3 shows a schematic, plan view of an embodiment of the microfluidic heat exchange system 110, which is configured to achieve pasteurization of a liquid (such as water) flowing through the microfluidic heat exchange system without the need for a second fluid stream to add heat to or remove heat from the liquid. FIG. 3 is schematic and it should be appreciated that variations in the actual configuration of the flow pathway, such as size and shape of the flow pathway, are possible. Embodiments of microfluidic heat exchange systems are described in detail in U.S. patent application Ser. No. 12/795,382, filed Jun. 7, 2010 and U.S. Patent Application Publication No. 2010/0326916, filed Jun. 7, 2010, which are each incorporated by reference in their entireties.

As described more fully below, the microfluidic heat exchange system defines a fluid flow pathway that can include (1) at least one fluid inlet; (2) a heater region where incoming fluid is heated to a pasteurization temperature via at least one heater; (3) a residence chamber where fluid remains at or above the pasteurization temperature for a predetermined time period; (4) a heat exchange section where incoming fluid receives heat from hotter (relative to the incoming fluid) outgoing fluid, and the outgoing fluid cools as it transfers heat to the incoming fluid; and (5) a fluid outlet where outgoing fluid exits in a cooled, pasteurized state. Depending on the desired temperature of the outgoing fluid, one or more additional heat exchanges may be used downstream to adjust the actual temperature of the outgoing fluid to the desired temperature for use, for example, in dialysis. This is especially true in warmer climates, where incoming water may be tens of degrees higher than water supplied in colder climates, which will result in higher outlet temperatures than may be desired unless further cooling is applied.

In an embodiment, the flow pathway is at least partially formed of one or more microchannels, although using microfluidic flow fields for portions of the fluid flow pathway such as the heat exchange section is also within the scope of the invention. The relatively reduced dimensions of a microchannel enhance heat transfer rates of the heat exchange system by providing a reduced diffusional path length and amount of material between counterflow pathways in the system. In an embodiment, a microchannel has at least one dimension less than about 1000 μm. The dimensions of a microchannel can vary and are generally engineered to achieve desired heat transfer characteristics. A microchannel in the range of about 0.1 to about 1 mm in hydraulic diameter generally achieves laminar fluid flow through the microchannel, particularly in a heat exchange region of the microchannel. The small size of a microchannel also permits the heat exchange system 110 to be compact and lightweight. In an embodiment, the microchannels are formed in one or more laminae that are arranged in a stacked configuration.

The flow pathway of the microfluidic heat exchange system 110 may be arranged in a counterflow pathway configuration. The flow pathway can be arranged such that cooler, incoming fluid flows in thermal communication with hotter, outgoing fluid. The hotter, outgoing fluid transfers thermal energy to the colder, incoming fluid to assist the heaters in heating the incoming fluid to the pasteurization temperature. This internal preheating of the incoming fluid to a temperature higher than its temperature at the inlet reduces the amount of energy used by the heaters to reach the desired peak temperature. In addition, the transfer of thermal energy from the outgoing fluid to the incoming fluid causes the previously heated, outgoing fluid to cool prior to exiting through the fluid outlet. Thus, the fluid is “cold” as it enters the microfluidic heat exchange system 110, is then heated (first via heat exchange and then via the heaters) as it passes through the internal fluid pathway, and is “cold” once again as it exits the microfluidic heat exchange system 110. The fluid can enter the microfluidic heat exchange system 110 at a first temperature and is heated (via heat exchange and via the heaters) to a second temperature that is greater than the first temperature. As the fluid follows an exit pathway, the fluid (at the second temperature) transfers heat to incoming fluid such that the fluid drops to a third temperature that is lower than the second temperature and that is higher than the first temperature.

Exemplary embodiments of a fluid pathway and corresponding components of the microfluidic heat exchange system 110 are now described in more detail with reference to FIG. 3, which depicts a bayonet-style heat exchanger, with the inlet and outlet on one side of the device, a central heat exchange portion, and a heating section toward the opposite end. The fluid can enter the microfluidic heat exchange system 110 through an inlet 282. In the illustrated embodiment, the flow pathway can branch into one or more inflow microchannels 284 that are positioned in a counterflow arrangement with an outflow microchannel 286. As mentioned, microfluidic heat exchange system 110 may be formed by a stack of layered laminae. The inflow microchannels 284 may be positioned in separate layers with respect to the outflow microchannels 286 such that inflow microchannels 284 are positioned above or below the outflow microchannels 286 in an interleaved fashion. In another embodiment, the inflow microchannels 284 and outflow microchannels 286 are positioned on a single layer.

The outflow microchannel 286 can communicate with an outlet 288. In the illustrated embodiment, the inlet 282 and outlet 288 are positioned on the same end of the microfluidic heat exchange system 110, although the inlet 282 and outlet 288 may also be positioned at different positions relative to one another. The counterflow arrangement places the inflow microchannels 284 in thermal communication with the outflow microchannel 286. In this regard, fluid in the inflow microchannels 284 may flow along a directional vector that is oriented about 180 degrees to a directional vector of fluid flow in the outflow microchannels 286. The inflow and outflow microchannels may also be in a cross flow configuration wherein fluid in the inflow microchannels 284 may flow along a directional vector that is oriented between about 180 degrees to about 90 degrees relative to a directional vector of fluid flow in the outflow microchannels 286. The orientation of the inflow microchannels relative to the outflow microchannels may vary in any matter that is configured to achieve the desired degree of thermal communication between the inflow and outflow microchannels.

One or more heaters 292 can be positioned in thermal communication with at least the inflow microchannels 284 such that the heaters 292 can provide heat to fluid flowing in the system. The heaters 292 may be positioned inside the inflow microchannels 284 such that fluid must flow around multiple sides of the heaters 292. Or, the heaters 292 may be positioned to the side of the inflow microchannels 284 such that fluid flows along one side of the heaters 292. In any event, the heaters 292 can transfer heat to the fluid sufficient to cause the temperature of the fluid to achieve a desired temperature, which may include a pasteurization temperature in the case of water to be purified. In an embodiment, the fluid is water and the heaters 292 assist in heating the fluid to a temperature of at least 100 degrees Celsius at standard atmospheric pressure. In an embodiment, the fluid is water and the heaters 292 assist in heating the fluid to a temperature of at least 120 degrees Celsius. In an embodiment, the fluid is water and the heaters 292 assist in heating the fluid to a temperature of at least 130 degrees Celsius. In an embodiment, the fluid is water and the heaters 292 assist in heating the fluid to a temperature of at least 138 degrees Celsius. In another embodiment, the fluid is water and is heated to a temperature in the range of about 138 degrees Celsius to about 150 degrees Celsius. In another embodiment, the fluid is heated to the highest temperature possible without achieving vaporization of the fluid.

Thus, the microfluidic heat exchange system 110 may maintain the fluid as a single phase liquid. Because water typically changes phases from a liquid into a gaseous state around 100 degrees Celsius, the heat exchange system can be pressurized such that the heating water at the temperatures set forth above are maintained at single-phase liquid throughout. Pressures above the saturation pressure corresponding to the highest temperature in the heat exchange system are sufficient to maintain the fluid in a liquid state. As a margin of safety, the pressure can be kept at 10 psi or higher above the saturation pressure. In an embodiment, the pressure of water in the microfluidic heat exchange system is maintained greater than 485 kPa to prevent boiling of the water, and may be maintained significantly in excess of that level, such as 620 kPa or even as high as 900 kPa, in order to ensure no boiling occurs. These pressures can be maintained in the heat exchange system using a pump and a throttling valve or a fixed flow controlling oriface. A pump upstream of the heat exchange system and a throttling valve downstream of the heat exchange system can be used where the pump and throttling valve operate in a closed loop control setup (such as with sensors) to maintain the desired pressure and flow rate throughout the heat exchange system.

Once the fluid has been heated to the pasteurization temperature, the fluid can pass into a residence chamber 294 where the fluid remains heated at or above the pasteurization temperature for a predetermined amount of time, referred to as the “residence time”, or sometimes referred to as the “dwell time”. In an embodiment, the dwell time can be less than or equal to one second, between one and two seconds, or at least about two seconds depending on the flow path length and flow rate of the fluid. Higher temperatures are more effective at killing bacteria and shorter residence times mean a more compact device. Ultrahigh temperature pasteurization, that is designed to kill all Colony Forming Units (CFUs) of bacteria in a solution containing up to a concentration of 10⁻⁶ CFU/ml (such as for purifying the water for use with infusible dialysate), is defined to be achieved when water is heated to a temperature of 138 degrees Celsius to 150 degrees Celsius for a dwell time of at least about two seconds. Ultrapure dialysate has a bacterial load no greater than 0.1 CFU/ml. Table 1 (shown in the attached figures) indicates the required temperature and residence time to achieve various levels of pasteurization. The heat exchange system described herein is configured to achieve the various levels of pasteurization shown in Table 1.

The fluid can then flow from the residence chamber 294 to the outflow microchannel 286, where it flows toward the fluid outlet 288. As mentioned, the outflow microchannel 286 can be positioned in a counterflow relationship with the inflow microchannel 284 and in thermal communication with the inflow microchannel 284. In this manner, outgoing fluid (flowing through the outflow microchannel 286) thermally communicates with the incoming fluid (flowing through the inflow microchannel 284). As the heated fluid flows through the outflow microchannel 286, thermal energy from the heated fluid transfers to the cooler fluid flowing through the adjacent inflow microchannel 284. The exchange of thermal energy results in cooling of the fluid from its residence chamber temperature as it flows through the outflow microchannel 286. Moreover, the incoming fluid is preheated via the heat exchange as it flows through the inflow microchannel 284 prior to reaching the heaters 292. In an embodiment, the fluid in the outflow microchannel 284 is cooled to a temperature that is no lower than the lowest possible temperature that precludes bacterial infestation of the fluid. When the heat exchange system pasteurizes the fluid, bacteria in the fluid down to the desired level of purification are dead as the fluid exits the heat exchange system. In such a case, the temperature of the fluid after exiting the heat exchange system may be maintained at room temperature before use in dialysis. In another embodiment, the fluid exiting the heat exchange system is cooled to a temperature at or below normal body temperature.

Although an embodiment is shown in FIG. 3 as having an outlet channel sandwiched between an inflow channel, other arrangements of the channels are possible to achieve the desired degrees of heating and cooling and energy requirements of the heaters. Common to all embodiments, however, is that all fluid pathways within the system are designed to be traveled by a single fluid, without the need for a second fluid to add heat to or remove heat from the single fluid. In other words, the single fluid relies on itself, at various positions in the fluid pathway, to heat and cool itself.

The dimensions of the microfluidic heat exchange system 110 may vary. In an embodiment, the microfluidic heat exchange system 110 is sufficiently small to be held in the hand of a user. In another embodiment, the microfluidic heat exchange system 110 is a single body that weighs less than 5 pounds when dry. In another embodiment, the microfluidic heat exchange portion 350 of the overall system 110 has a volume of about one cubic inch. The dimensions of the microfluidic heat exchange system 110 may be selected to achieve desired temperature and dwell time characteristics.

As mentioned, an embodiment of the microfluidic heat exchange system 110 is made up of multiple laminar units stacked atop one another to form layers of laminae. A desired microfluidic fluid flow path may be etched into the surface of each lamina such that, when the laminae are stacked atop one another, microfluidic channels or flow fields are formed between the lamina. Furthermore, both blind etching and through etching may be used for forming the channels in the laminae. In particular, through etching allows the fluid to change the plane of laminae and move to other layers of the stack of laminae. This occurs in one embodiment at the outlet of the inflow laminae where the fluid enters the heater section, as described below. Through etching allows all laminae around the heater section to participate in heating of the fluid instead of maintaining the fluid only in the plane of the inlet laminae. This embodiment provides more surface area and lower overall fluid velocity to facilitate the heating of the fluid to the required temperature and ultimately contributes to the efficiency of the device.

The microchannels or flow fields derived from blind and/or through etching of the laminae form the fluid flow pathways. The microchannels and flow fields described herein can be at least partially formed of one or more microfluidic flow fields as disclosed in U.S. patent application Ser. No. 12/795,382, filed Jun. 7, 2010, and U.S. Patent Application Publication No. 2010/0326916, filed Jun. 7, 2010, which are both incorporated herein by reference in their entireties.

B. Dialysate Preparation Sub-System

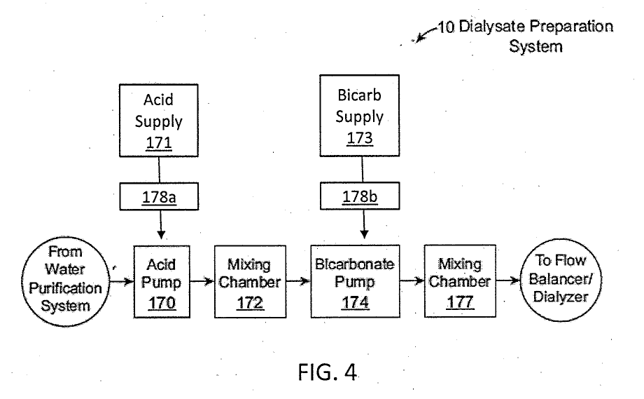

The water is in a pasteurized state as it exits the water purification system 5 and flows into the dialysate preparation system 10. The dialysate preparation system 10 is configured to mix the pasteurized water with a supply of concentrate solutions in order to make dialysate. FIG. 4 shows a high level, schematic view of the dialysate preparation system 5. The embodiment of FIG. 4 is exemplary and it should be appreciated that variations are within the scope of this disclosure.

The dialysate preparation system 10 can include an acid pump 170 that fluidly communicates with an acid supply 171 of concentrated acidified dialysate concentrate for mixing with the purified water. The water can flow from the water purification system 5 to the acid pump 170, which pumps the acid concentrate into the water. The water (mixed with acid) can then flow into a first mixing chamber 172, which is configured to mix the water with the acid such as by causing turbulent flow. From the first mixing chamber 172, the acid-water mixture can flow toward a bicarbonate pump 174. A sensor, such as a conductivity sensor, may be positioned downstream of the first mixing chamber 172. The conductivity sensor is configured to detect a level of electrolytes in the mixture. The conductivity sensor may be in a closed loop communication with the acid pump 170 and a control system that may regulate the speed of the acid pump 170 to achieve a desired level of acid pumping into the water.

The bicarbonate pump 174 can pump bicarbonate concentrate from a supply 173 into the acid-water mixture at a level sufficient to form dialysate. The resulting mixture of fluid flows into a second mixing chamber 177 and exits the second mixing chamber 177 as dialysate. Another sensor, such as a conductivity sensor, may be positioned downstream of the second mixing chamber 172. The second conductivity sensor may be in a closed loop communication with the bicarbonate pump 177. The dialysate can then flow toward the flow balancer system and the dialyzer.

In an embodiment, a pressure transducer 178a is positioned downstream of the acid supply 171 and upstream of the acid pump 170. A second pressure transducer 178b is positioned downstream of the bicarbonate supply 173 and upstream of the bicarbonate pump 174. The pressure transducers 178a and 178b are configured to measure fluid pressure along the fluid flow line. The pressure transducers 178 may be communicatively linked to a control system, such as in a feedback control relationship. The control system may use the pressure measurements of the pressure transducers 178 as an indication of whether one or both of the acid supply 171 and/or the bicarb supply 173 contains sufficient supply of acid or bicarb to continue a particular treatment. The control system periodically or continuously monitors the values reported by the pressure transducers and compares them with one or more threshold values. When a pressure is lower than a particular threshold, this is an indication that one or both of the supplies 171, 173 are empty, close to empty or due to become empty during a treatment schedule. Should the measured pressure be below the threshold, the control system then initiates a corresponding action, such as to issue an alert to a user. In another embodiment, the control system may provide an alert to the user as to the present status of the supplies, even if not yet close to empty, either by an indicator showing one or more fractional readings or a color of light indicating their status (e.g., green for sufficient supply, yellow for sufficient but low supply, red for insufficient supply). In another embodiment, the control system may prevent operation of the dialysis system if the level of one of the supplies 171 or 173 is sufficiently low to prevent a dangerous or undesirable operation for the user.

C. Dialyzer Sub-System

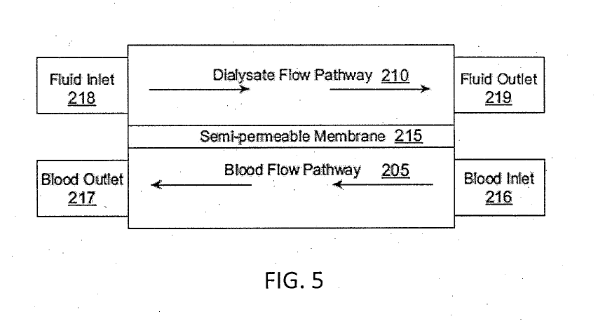

FIG. 5 is a schematic, cross-sectional view of the dialyzer 15, which defines a blood compartment having a blood flow pathway 205 and a dialysate compartment having a dialysate flow pathway 210 separated by a transfer layer comprised of a semi-permeable membrane 215. In an embodiment, the dialyzer includes one or more microfluidic pathways such as micro flow fields and/or microchannels. Exemplary embodiments of dialyzers that utilize micro flow fields and/or microchannels and/or flow field dialyzers are described in U.S. Patent Publication No. 2010/0326914, filed Jun. 7, 2010, which is incorporated by reference in its entirety. However, the dialysis system can be used with any of a variety of dialyzers including a variety of commercially-available dialyzers.

The blood (from a patient) can enter the blood flow pathway 205 via a blood inlet 216, flow through the blood flow pathway 205, and exit via a blood outlet 217. The dialysate can enter the dialysate flow pathway 210 via a fluid inlet 218, flow through the dialysate flow pathway 210, and exit via a fluid outlet 219. The semi-permeable membrane 215 is configured to allow the transfer of one or more substances from the blood in the blood flow pathway 205 to the dialysate in the dialysate flow pathway 210, or visa-versa.

Some examples of materials that may be used as the semipermeable membrane 215 include polymers, copolymers, metals, ceramics, composites, and/or liquid membranes. One example of a composite membrane is polysulfone-nanocrystalline cellulose composite membrane such as AN69 flat sheet membranes available from Gambro Medical. Gas-liquid contactor membranes may also be employed for transferring a substance between a liquid and gas such as for oxygenation of blood, whereby the membrane allows transfer of carbon dioxide and oxygen, such that oxygen transfers to blood from oxygen or oxygen-enriched air, and carbon dioxide transfers from the blood to the gas. Fluid membranes may also be employed. Fluid membranes can include a lamina having through cut microchannels containing fluid and a first and second membrane support positioned to contain fluid in the microchannels.

When flowing through the dialyzer 15, the blood and the dialysate may flow in a counter-flow configuration wherein blood flows through the blood flow pathway 205 in one direction and the dialysate flows through the dialysate flow pathway 210 in the opposite direction. The dialyzer 15 is described in the context of having a counter-flow configuration although a cross-flow configuration may also be used. As the blood and water flow along the membrane 215, hemodialysis can occur. The dialyzer 15 is also configured to perform ultrafiltration wherein a pressure differential across the membrane 215 results in fluid and dissolved solutes passing across the membrane 215 from the blood to the dialysate.

The dialyzer 15 is also configured to perform hemodiafiltration wherein solute movement across the semipermeable membrane 215 is governed by convection rather than by diffusion. A positive hydrostatic pressure differential between the blood flow pathway 205 and the dialysate flow pathway 210 drives water and solutes across the semipermeable membrane 215 from the blood flow pathway to the fluid flow pathway. Solutes of both small and large molecules get dragged through the semipermeable membrane 215 along with the fluid. In a typical hemodiafiltration procedure, the direction of water and solute movement can oscillate between moving water and solutes from the blood into the dialysate and moving water and solutes from the dialysate into the blood. Over a predetermined span of time, there is a net zero loss and zero net gain of fluid from the blood into the dialysate. However, during discrete time periods within that span of time, there can be a net loss of fluid from the blood into the dialysate and a net gain of fluid into the blood from the dialysate.

A possible complication of dialysis is an electrolyte imbalance and changes in the extracellular fluid volume that can ultimately cause more serious issues including cardiac problems if left unchecked or untreated. Electrolytes can diffuse from areas of high concentration to areas of low concentration until equilibrium is reached. For example, in some circumstances, patient blood can have higher sodium content than the dialysate solution which can result in a negative sodium balance during dialysis. Sodium and water exit the blood towards the dialysate resulting in a decrease in patient extracellular fluid volume leading to hypotension and associated cardiac changes. The higher sodium content in a patient's blood can also cause an osmotic gradient that results in the blood tending to pull fluid from the dialysate into the blood stream making it more difficult to remove fluid during the dialysis treatment. The patient's sodium level can be reduced to below their individual “sodium set point” resulting in the patient craving salt following a treatment and can lead to lower fluid intake between dialysis treatments. In other circumstances, patient blood can have lower sodium content than the dialysate solution that can result in a positive sodium balance during dialysis. Sodium and water enter the blood from the dialysate resulting in an increase in extracellular fluid volume leading to hypertension and associated cardiac changes. The patient's sodium level can be increased to above their individual “sodium set point” resulting in the patient craving fluid after the treatment that can lead to higher fluid intake between dialysis treatment. As such, controlling the dialysis, and the makeup of the dialysate, in such a way so as not to inadvertently affect body sodium or other electrolyte content and extracellular fluid volume to prevent the aforementioned conditions may be desirable as it would improve dialysis patient health and long-term survival.

The systems described herein can control body sodium content in real-time by matching an indirectly measured conductivity of patient blood with a directly measured conductivity of dialysate prior to and after flow through the dialyzer. In this regard, the system is configured to measure a conductivity of the dialysate prior to contact with the patient's blood. The system stores the measured conductivity in a memory for real-time comparison with conductivity of dialysate measured after contact with blood using a microprocessor. The comparison provides an indication of how much sodium is diffusing out of (or into) the dialysate during dialysis. Plasma electrical correlates with plasma sodium. Electrolyte concentration of the dialysate can be measured upstream and downstream of the dialyzer. In some embodiments, total electrolyte concentration can be measured by a conductivity sensor positioned upstream of the dialyzer in the new dialysate line and a second conductivity sensor positioned downstream of the dialyzer in the used dialysate line. The difference of the concentrations is a direct indicator of the transfer rate of electrolytes, sodium in particular, between the dialysate and the blood. The control system is configured to adjust dialysate concentration in real-time until the transfer rate of electrolytes disappears or falls below a predetermined threshold. A difference equal to zero would indicate that a sodium level equilibrium has been achieved between the patient blood and dialysate. Measuring conductivity and flow of the dialysate provides a more accurate reading than attempting to perform similar measurements on the blood-side such as by performing biochemical analysis of patient sodium levels.

D. Flow Balancer System

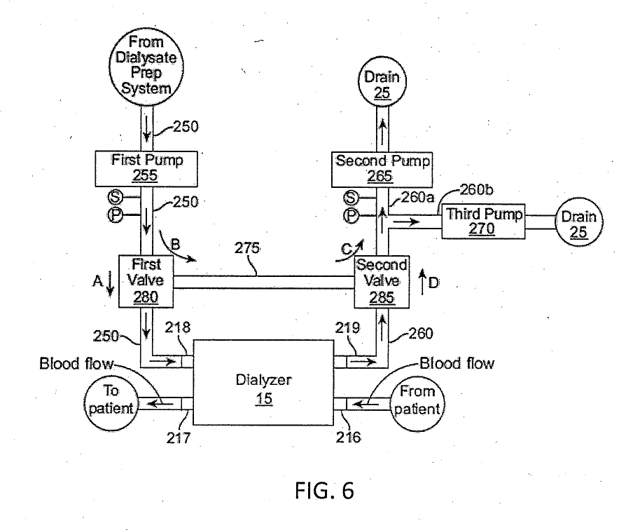

FIG. 6 shows a schematic view of the flow balancer system 20 including the dialyzer 15. The flow balancer system 20 is adapted to regulate the flow of dialysate into and out of the dialyzer 15 to achieve various types of dialysis, including hemodialysis, ultrafiltration, and hemodiafiltration. The flow balancer system 20 can include a first pump for pumping dialysate into a dialyzer and a second pump for pumping dialysate out of the dialyzer. The system can also include a third pump that provides improved control of a level of ultrafiltration, hemodiafiltration, or both. By varying the relative pump speeds of the pumps, an operator can vary the level of blood filtration and can also selectively achieve ultrafiltration and hemodiafiltration of the blood.

The flow balancer system 20 can include plumbing that forms a plurality of fluid flow pathways, which may be any type of conduit through which a fluid such as dialysate may flow. The fluid flow pathways can include an inlet pathway 250 through which a fluid such as unused dialysate flows from the dialysate preparation system 10 toward and into the dialyzer 15. At least a first pump 255 is positioned along or in communication with the inlet pathway 250 for pumping the fluid toward the dialyzer 15 at a desired flow rate. One or more sensors S may be coupled to the fluid flow pathway for sensing one or more characteristics of the incoming fluid, such as pressure, flow rate, temperature, conductivity, etc. In addition, one or more sample ports P may be coupled to the fluid flow pathways that provide access to fluid flowing through the piping. FIG. 6 shows the sensors S and sample ports P coupled to the fluid flow pathways at specific locations, although the quantity and locations of the sensors S and sample ports P may vary.

The fluid flow pathways can further include an outlet pathway 260 through which used dialysate flows out of the dialyzer 15 toward one or more drains 25. In some embodiments, the dialysate exiting the dialyzer may be used to pre-heat other incoming fluids in the system, such as the water stream entering the heat exchange and purification system, before reaching the drain 25. The outlet pathway 260 can bifurcate into two or more outlet pathways including a main outlet pathway 260a and a secondary outlet pathway 260b. At least a second pump 265 can be positioned along or in communication with the main outlet pathway 260a for pumping the dialysate out of and away from the dialyzer 15 through the main outlet pathway 260a.

A third pump 270 can be positioned along or in communication with the secondary outlet pathway second valve 285. The third pump 270 can be used to augment fluid flow through the fluid flow pathways such as to selectively achieve differentials in flow rates between the inlet pathway 250 and the outlet pathway 260 pursuant to achieving various types of dialysis, including hemodialysis, ultrafiltration, and hemodiafiltration, as described more fully below. The third pump can pump dialysate through the fluid flow pathways when the system is in dialysis mode. The third pump may also pump another fluid, such as water or disinfectant, when the system is in a different mode, such as in a calibration mode or in a cleaning mode.

The third pump 270 can be positioned along the inlet pathway 250 upstream of the inlet 218 of the dialyzer 15. In this embodiment, the secondary outlet pathway 260 branches off the inlet pathway 250 at a location downstream of the first pump 255 and upstream of the first valve 280. The third pump 270 can pump fluid toward the drain 25. In another embodiment, the third pump 270 and the second pump 265 are both positioned along a single, non-bifurcating outflow pathway.

Various types of pumps may be used for the first, second and third pumps. In an embodiment, the pumps are nutating pumps. On other embodiments, the pumps could be rotary lobe pumps, progressing cavity pumps, rotary gear pumps, piston pumps, diaphragm pumps, screw pumps, gear pumps, hydraulic pumps, vane pumps, regenerative (peripheral) pumps, or peristaltic pumps, or any combination thereof. Other types of pumps can also be used. The first pump 255 and the second pump 265 may be driven by a common shaft to ensure synchrony of the pump strokes and the volume of fluid pumped. It is understood that first pump 255 and the second pump 265 may also be fully independent from each other.

As mentioned, any of a variety of fluid conduits may be used to form the fluid flow pathways of the flow balancer system 20. In an embodiment, at least a portion of the fluid flow pathway is formed of piping having an inside diameter from ⅛ inch to ½ inch. The flow rate in the piping could range between about 50 ml/min to about 1,000 ml/min. In an embodiment, the flow rate is in the range of between about 100 ml/min and about 300 ml/min.

The fluid flow pathways further can include a bypass pathway 275 that directly connects fluidly the inlet pathway 250 and the outlet pathway 260. An exemplary purpose of the bypass pathway 275 is to provide a fluid flow pathway where fluid can flow into and out of the dialysis system and bypass the dialyzer 15, such as for flushing, cleaning or calibrating the system. In an embodiment, the junction between the inlet pathway 250 and bypass pathway 275 is located upstream of the fluid inlet 120 of the dialyzer 15, and the junction between the bypass pathway 275 and the outlet pathway is located downstream of the fluid outlet 125 of the dialyzer 15. However, other configurations of the bypass pathway 275 can be used to achieve bypassing of the dialyzer 15.

A first valve 280 can be positioned at the junction between the inlet pathway 250 and the bypass pathway 275. A second valve 285 can be positioned at the junction between the bypass pathway 275 and the outlet pathway 260. The first valve 280 and second valve 285 can be three-way valves, such as solenoid valves, that can be used to selectively regulate fluid flow through the fluid flow pathways. That is, the first valve 280 can be set to either of two or more settings including (1) a dialysis setting wherein the first valve directs all incoming fluid along the inlet pathway 250 toward the dialyzer 15 (as represented by arrow A in FIG. 6) and prevents incoming fluid from flowing into the bypass pathway 275; or (2) a bypass setting wherein the first valve 280 diverts all the incoming fluid into the bypass pathway 275 (as represented by arrow B in FIG. 6) and the prevents incoming fluid from flowing past the first valve toward the dialyzer 15.

The second valve 285 can also be set to either of two settings including (1) a bypass setting wherein the second valve 285 directs incoming fluid from the bypass pathway 275 into the outlet pathway 260 (as represented by arrow C in FIG. 6); or (2) a dialysis setting wherein the second valve 285 closes flow from the bypass pathway 275 such that outgoing fluid from the dialyzer outlet 125 continues to flow outward along the outlet pathway 260 (as represented by arrow D in FIG. 6.) The first valve 280 and the second valve 285 are generally both set in tandem to either the bypass setting or the dialysis setting. The system may include a control and safety system that ensures that the first and second valves are not set to incompatible settings.

The arrangement of the various components of the dialysis system shown in FIG. 6 is exemplary and other arrangements are possible. For example, the flow pathways and the pumps may be placed in different locations along the flow pathways from what is shown in FIG. 6. In an embodiment, the third pump 270 can be positioned in the flow pathway at a location upstream of the dialyzer 15 and downstream of the first valve 280 or the third pump can be positioned downstream of the dialyzer 15 and upstream of the second valve 285. Moreover, the system can employ more than three pumps.

Operation of the Flow Balancer System to Achieve Hemodialysis

With reference again to FIG. 6, the flow balancer system 20 achieves hemodialysis without ultrafiltration when the flow rate through the inlet pathway 250 is equal to or substantially equal to the flow rate through the outlet pathway 260. In other words, hemodialysis without ultrafiltration is achieved where the amount of dialysate flowing into dialyzer 15 via the inlet pathway 250 is substantially equal to the amount of dialysate flowing out of the dialyzer via the outlet pathway 260 over a period time. This can be achieved by operating the first pump 255 at a first pump rate to provide a first flow rate through the inlet pathway 250 and operating the second pump 265 and the third pump 270 at respective pump rates that collectively achieve a flow rate through the outlet pathway 260 that is equal to the flow rate through the inlet pathway 250.

In an embodiment, the system performs a hemodialysis procedure utilizing all three pumps in an active state substantially continuously throughout the hemodialysis procedure. The system adjusts the pump rate of the third pump 270 to achieve a desired balance of equal flow rates between the inlet pathway 250 and the outlet pathway 260. In this embodiment, the first pump 255, second pump 265, and third pump 270 are all active throughout the hemodialysis procedure with the first and second pumps operating at different pump rates and the third pump operating at a pump rate that achieves a balanced flow rate between the inlet pathway 250 and the outlet pathway 136. The third pump is typically operated at a pump rate that is equal to the differential between the pump rate of the first pump and the pump rate of the second pump. In this manner, the second and third pumps collectively achieve a flow rate through the outlet pathway 260 that is equal to the flow rate through the inlet pathway 250.

For example, to achieve a desired flow rate of, for example, 100 ml/min through the dialyzer, the first pump 255 is set to provide a flow rate of 100 ml/min through the inlet pathway 250 and the second pump 265 is deliberately set out of balance with the first pump 255, to provide, for example, a flow rate of only 80 ml/min. This would provide a flow rate differential of 20 ml/min between the first pump and the second pump. The pump rate of third pump 270 is set to provide a flow rate of 20 ml/min, which is equal to the differential between the flow rates of the first and second pumps. In this manner, the second pump 265 and the third pump 270 collectively achieve a flow rate of 100 ml/min through the outlet pathway 260 which is equal to the flow rate of through the inlet pathway 250 such that the flow rates are balanced across the dialyzer. Under such conditions, waste solutes move across the dialyzer's semipermeable membrane from the blood stream into the dialysate via diffusion to perform hemodialysis.

The flow rates through the inlet pathway 250 and the outlet pathway 260 may be measured using one or more of the sensors S. In an embodiment, the sensors are flow rate sensors that directly measure flow rates through the inlet pathway 250 and outlet pathway 260. In another embodiment, the sensors are pressure sensors that provide indications as to the fluid pressure within the inlet pathway 250 and the fluid pressure within the outlet pathway 260. Fluid pressure is a function of the flow rate through the flow pathways and therefore provides an indirect measurement of flow rate. Where the fluid pressure in the inlet pathway 250 is equal to the fluid pressure in the outlet pathway 260, this is an indication that the flow rates are balanced between the inlet pathway and outlet pathway. Where the fluid pressure in the inlet pathway 250 is less than the fluid pressure through the outlet pathway 260, this is an indication that the flow rate through the inlet pathway 250 is less than the flow rate through the outlet pathway 260. Where the fluid pressure in the inlet pathway 250 is greater than the fluid pressure through the outlet pathway 260, this is an indication that the flow rate through the inlet pathway 250 is greater than the flow rate through the outlet pathway 260. The system of fluid pathways may include one or more damping mechanisms for dampening any extreme fluctuations in pressure within the fluid pathways.

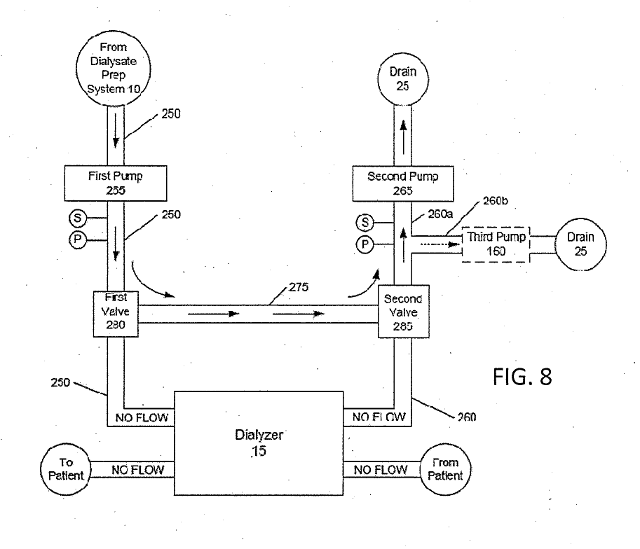

In the latter two situations, the pump rate of the third pump 270 may be adjusted in response to a pressure differential between the inlet and outlet pathways such as in a calibration procedure, to achieve a balanced flow rate between the inlet pathway 250 and outlet pathway 260. The calibration procedure may optionally be performed with the system in a calibration mode such that the first and second valves are set to cause fluid to flow through the bypass pathway 275 and bypass the dialyzer 15, as represented in FIG. 8 and described in more detail below. When the calibration procedure is performed by bypassing the dialyzer 15 and a pressure differential is detected between the inlet and outlet pathways, the flow of the third pump 270 may be appropriately adjusted ‘on the fly’ to increase or decrease the third pump's speed to achieve the desired flow rate in the outlet pathway 260 without having to turn the pump on or off. In this regard, the pressure sensors S and the three pumps, as well as the valves 280 and 285, may be connected in a closed loop control system to achieve automatic balancing of the flow rates.

In another embodiment, a balanced flow rate between the inlet pathway 250 and the outlet pathway 260 is achieved in theory at least by the first pump 255 and the second pump 265 operating at the same pump rate to achieve equal flow rates through the inlet pathway 250 and outlet pathway 260. Although it is theoretically possible to match the flow rates of the first pump 255 and the second pump 265, various factors may result in the actual fluid flow rate in the inlet pathway 250 differing from the actual fluid flow rate in the outlet pathway 260. The factors may include trapped air, hardware wear, and fluid leakage, which can cause the flow rates of the first and second pumps to diverge over time from a preset or desired value. Typical technologies in dialysis systems are unable to correct the flow balance for these types of factors.

Thus, there may come a time when a balanced flow rate cannot easily be achieved through use of the first and second pumps alone, and thus when there exists a need for correction to equalize the flow rates between the inlet pathway 250 and outlet pathway 260. Where the fluid flow rates are different, the third pump 270 can be used to correct the differing flow rates by being activated to pump fluid through the secondary outlet pathway 260b at a rate that is equal to the delta between the fluid flow rate through the inlet pathway 250 and the fluid flow rate through the outlet pathway 260. The system is preferably configured such that the first pump 255 is prevented from pumping less fluid than the second pump 265 such that the first pump 255 always pumps at a higher rate than the second pump 265. The system preferably includes a control system that detects a condition where the first pump 255 inadvertently pumps at a slower rate than the second pump 265 and sets off an alarm or moves the system out of dialysis mode if such a situation occurs.

According to a flow rate correction process, the sensors S (FIG. 6) are used to measure the flow rates through the inlet pathway 250 and the outlet pathway 260. A comparison is performed between the flow rate through the inlet pathway 250 and the flow rate through the outlet pathway 260. Where the flow rates are different, the third pump 270 is activated from a de-activated state to cause fluid to flow into the secondary outlet pathway second valve 285 at a rate selected to cause the overall flow rate in the outlet pathway 260 to be equal to the flow rate in the inlet pathway 250. A mechanism such as a servo mechanism may be used to adjust the stroke volume of the first pump 255 and/or the second pump 265 until balance of the flow rates is restored (as may be evidenced, for example, by the presence of the same fluid pressure in both the inlet pathway 250 and the outlet pathway 260).

As mentioned, the sensors S may be communicatively coupled to a control system and to the three pumps in a closed loop system. The control system includes hardware and/or software that automatically activates and/or deactivates the third pump 270 or adjusts the pump rate of the third pump 270 as needed in response to differences in detected flow rates from predetermined values or from each other, to equalize the flow rates between the inlet pathway 250 and outlet pathway 260. It should be appreciated that other measurements, such as fluid pressure in the inlet and outlet pathways, may be used to indirectly calculate the flow rates rather than directly measuring the flow rates. In this regard, the fluid pressures within the inlet pathway and the outlet pathway may be measured for any detectable change in pressure from a predetermined value or from each other. The flow pathways may be adapted to be essentially non-compliant so that a small difference in the flow rates of the first pump 255 and the second pump 265 will cause a rapid pressure change either negative or positive in magnitude.

The system may initially and/or periodically run in a calibration mode (sometimes also referred to as a UF checking mode) wherein a fluid (which may or may not be dialysate) is flowed through the flow pathways with the first valve 280 and second valve 285 set to the “bypass setting” such that fluid flowing through the system bypasses the dialyzer 15 via the bypass pathway 275. FIG. 8 shows a schematic representation of the system running in such a calibration mode where the dialyzer 15 is bypassed. In the embodiment where the system utilizes all three pumps in an active state substantially continuously throughout the hemodialysis procedure, the first and second pumps are initially deliberately set to achieve unbalanced flow rates. The sensors S in the flow pathway are then used to measure the fluid flow rate or pressure through the inlet pathway and the fluid flow rate or pressure through the outlet pathway. The third pump 270 is then set at a pump speed that achieves a substantially balanced flow rate between the inlet pathway 250 and outlet pathway 260.

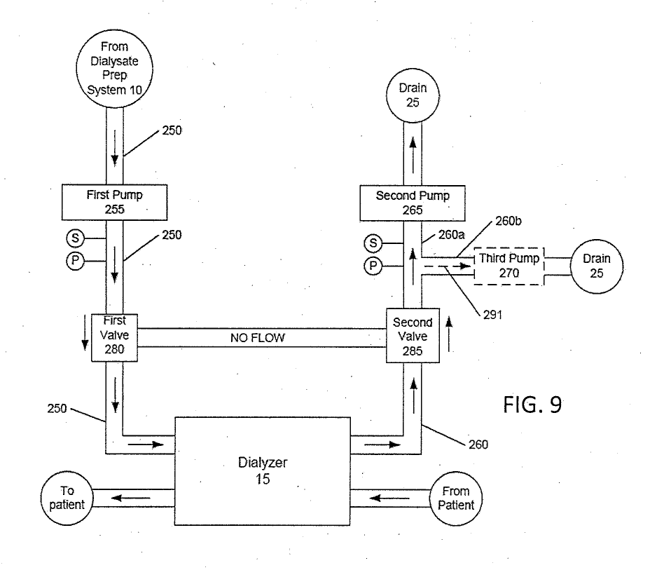

In the other embodiment, the first pump 255 and second pump 265 are initially set to achieve equal flow rates without necessarily requiring the assistance of the third pump 270, which is initially inactive. The sensors S in the flow pathway are then used to measure the fluid flow rate through the inlet pathway and the fluid flow rate through the outlet pathway. Where the fluid flow rates are equal, the third pump 270 remains inactive. However, where the fluid flow rates are not equal, the third pump 270 is run at a rate that compensates for the discrepancy in flow rates between the inlet pathway 250 and outlet pathway 260. As mentioned, the third pump 270 may operate in a closed-loop relationship with the flow rate sensors and/or the pressure sensors. FIG. 9 shows the third pump 270 in phantom lines to represent the third pump may or may not be activated depending on whether there is a flow rate differential between the inlet pathway 250 and outlet pathway 260. The calibration procedure that does not require activating and de-activating the third pump is preferred as the system may run more efficiently when all three pumps are continuously operating.

After the calibration procedure is completed, the valves 280 and 285 may be set to the “dialysis setting” such that fluid flows from the source 110, through the inlet pathway 250, into the dialyzer 15, out of the dialyzer, and into the outlet pathway 260 from the dialyzer 15. When configured as such, the system can be used for dialysis by flowing dialysate into and out of the dialyzer 15 via the inlet and outlet pathways, and by also by flowing blood into and out of the dialyzer. During dialysis, the previously described calibration procedure may be periodically repeated, such as at predetermined intervals, to ensure that the flow rates of the inlet and outlet pathways remain within desired ranges.

In an embodiment, calibration is run only at the beginning of a dialysis session. In a more preferred embodiment, calibration is run periodically during the dialysis session, to ensure that the desired flow balance is maintained throughout the session. The control system can cycle the valves 280 and 285 controlling incoming flow stream between the dialysis setting and the bypass setting and run the calibration steps without additional interruptions to the dialysis session. During the calibration process, when the dialysate fluid bypasses the dialyzer 15, dialysis of the blood that passes through the dialyzer during that period of time is unhampered due to no fresh dialysate being provided to the dialyzer 15, though the blood may cool slightly. As long as the calibration step can be conducted over a relatively short period of time relative to the time between calibrations, the calibration has no material effect on the quality of dialysis being provided to the patient. In an embodiment, the dialysis system can be cycled between calibration for one minute followed by 60 minutes of dialysate being delivered through the dialyzer. In another embodiment, the dialysis system can be cycled between calibration for 30 seconds followed by 120 minutes of dialysate being delivered through the dialyzer.

FIG. 9 schematically shows the system running in a dialysis mode. The third pump 270 and the flow arrow 291 through the secondary outlet pathway second valve 285 are shown in phantom lines to indicate that the third pump 270 may or may not be active while the system is in dialysis mode. The third pump 270 may be active in the situation where the third pump 270 is needed to equalize the flow rates between the inlet pathway and outlet pathways. Or, the flow rates of the inlet and outlet pathways may be equal without the assistance of the third pump 270, in which case the third pump 270 remains inactive.

Operation of the Flow Balancer System to Achieve Ultrafiltration

The dialysis system achieves ultrafiltration in the situation where the flow rate through the inlet pathway 250 differs from the flow rate through the outlet pathway 260 such that there is an unbalanced flow rate across the dialyzer. Where the flow rate through the outlet pathway 260 is greater than the flow rate through the inlet pathway 250, the dialyzer 15 pulls fluid from the blood across the semipermeable membrane by a convective process in order to compensate for the unbalanced flow rate. In an embodiment, the system utilizes all three pumps substantially continuously throughout the procedure and the pump rate of the third pump 270 is adjusted to achieve a desired flow rate differential between the inlet pathway 250 and the outlet pathway 260 to perform ultrafiltration. That is, the first pump 255, second pump 265, and third pump 270 are all active with the first and second pumps operating at different pump rates. The third pump is then operated at a pump rate that intentionally achieves a desired imbalance of flow rates between the inlet pathway 250 and the outlet pathway 136 sufficient to cause ultrafiltration.

For example, to achieve the removal of fluid at a rate 10 ml/min from the blood stream, the first pump 255 is set to provide a flow rate of 100 ml/min through the inlet pathway 250 and the second pump 265 is deliberately set out of balance with the first pump 255, to provide, for example, a flow rate of only 80 ml/min. The third pump 270 is then set to provide a flow rate of 30 ml/min such that the second and third pumps collectively provide a flow rate of 110 ml/min through the outlet pathway 260. With a flow rate of 100 ml/min through the inlet pathway 250 and a flow rate of 110 ml/min through the outlet pathway, the dialyzer 15 compensates for the 10 ml/min flow rate differential by transferring 10 ml/min of fluid from the blood stream into the dialysate.

In another example, to achieve the addition of fluid at a flow rate of 10 ml/min into the blood stream, the first pump 255 is set to provide a flow rate of 100 ml/min through the inlet pathway 250 and the second pump 265 is again deliberately set out of balance with the first pump 255, to provide, for example, a flow rate of only 80 ml/min. The third pump 270 is then set to provide a flow rate of only 10 ml/min such that the second and third pumps collectively provide a flow rate of 90 ml/min through the outlet pathway 260. With a flow rate of 100 ml/min through the inlet pathway 250 and a flow rate of 90 ml/min through the outlet pathway, there is a transfer of 10 ml/min from the dialysate into the blood stream in order to compensate for the flow rate differential. It should be appreciated that the flow rate values in the preceding examples and following examples are only for purpose of example and that the actual flow rates as well as the relative flow rates can vary to achieve a desired level of ultrafiltration or reverse ultrafiltration.

The speed of the third pump 270 can be varied to selectively vary an amount of ultrafiltration. For example, if it is determined that the ultrafiltration is greater than desired when pulling fluid out of the blood, for example, the pump speed of the third pump 270 can be slowed down, reducing the amount of extra fluid that the third pump 270 draws out of the dialyzer. Where the ultrafiltration is not great enough when compared against a desired predetermined value, the pump speed of the third pump 270 may be increased in the case where fluid is being pulled out of the blood into the dialysate, for example, to draw an even greater amount of fluid out of the dialyzer and, hence, the blood.

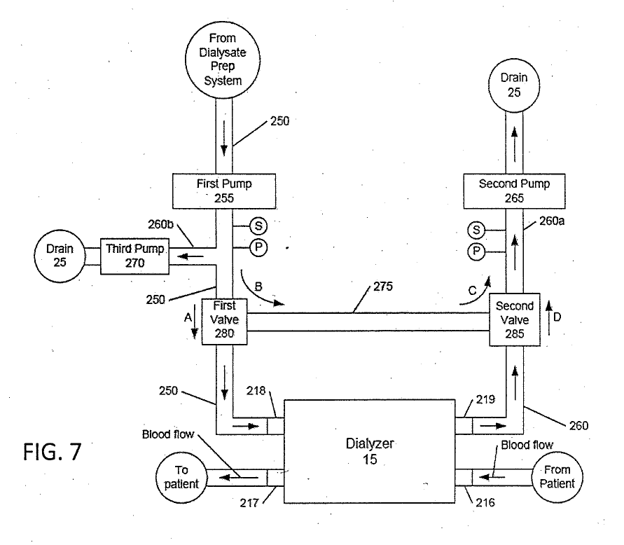

In another embodiment, the third pump 270 may be coupled to a source of fluid such that the third pump 270 outputs extra fluid into the flow pathway via the secondary outlet pathway second valve 285, such as in the embodiment of FIG. 7. The extra fluid introduced into the flow pathway is transferred across the semi-permeable membrane 215 into the blood.

Operation of the Flow Balancer System to Achieve Hemodiafiltration

The dialysis system is configured to achieve hemodiafiltration by oscillating the speed of the third pump between (1) a first speed such that the second and third pump collectively achieve a flow rate through the outlet pathway that is greater than the flow rate through the inlet pathway; and (2) a second speed such that the second and third pump collectively achieve a flow rate through the outlet pathway that is less than the flow rate through the inlet pathway. In this manner, the third pump 270 can be used to intermittently alternate the flow rate differential between a state where the dialyzer 15 pulls fluid from the blood stream into the dialysate and a state where the dialyzer 15 pushes fluid from the dialysate into the blood stream. Over a predetermined span of time, there should be a zero net loss (or substantially a zero net loss) of fluid from the blood and a zero net gain (or substantially a zero net gain) of fluid into the blood for the process of hemodiafiltration. However, during that span of time, the dialyzer 15 periodically transfers fluid into the blood from the dialysate and periodically transfers fluid out of the blood into the dialysate. If ultrafiltration is desired to be performed at the same time as the hemodiafiltration, then the pumps can be operated in such a way so that in addition to the cycling of fluid into and out of the blood over time, there also occurs a net gain or loss of fluid to or from the blood over a predetermined span of time.

For example over an exemplary time span of ten minutes, the first pump 255 is set to provide a flow rate of 100 ml/min through the inlet pathway 250 and the second pump 265 is again deliberately set out of balance with the first pump 255, to provide, for example, a flow rate of only 80 ml/min. The speed of pump 270 can be cycled between a rate of 10 ml/min for a period of 30 seconds and 30 ml/min for a period of 30 seconds. During the periods when the speed of the third pump 270 is at a rate of 10 ml/min, the total flow rate through the outlet pathway 260 is 90 ml/min with the flow rate through the inlet pathway 250 at 100 ml/min, resulting in an unbalanced flow rate that causes the dialyzer 15 to transfer 10 ml/min of fluid into the blood stream. During the periods when the speed of the third pump 270 is at a rate of 30 ml/min, the total flow rate through the outlet pathway 260 is 110 ml/min with the flow rate through the inlet pathway 250 at 100 ml/min, resulting in an unbalanced flow rate that causes the dialyzer 15 to transfer 10 ml/min of fluid from the blood stream into dialysate. Over the span of ten minutes with alternating periods of 30 seconds as described above, there is a net balanced flow rate of 100 ml/min across the dialyzer without any net addition or subtraction of fluid from the blood. This serves the purpose of passing fluid to the blood across the membrane and then fluid from the blood to the dialysate across the membrane to achieve hemodiafiltration of the blood and increases the removal of large-molecular waste products that would not otherwise be effectively dialyzed. In this way, operation of the three or more-pump system can achieve all of hemodialysis, ultrafiltration and hemodiafiltration through how the speeds of the first, second, and third pumps are controlled. This type of operation has heretofore not been possible in other dialysis systems.

In another embodiment, shown in FIG. 7, the third pump is located on the inlet flow side of the dialyzer instead of on the outlet flow path, such that the first and third pumps collectively achieve the desired inlet flow rate and the second pump achieves the desired outlet flow rate to perform one or more of hemodialysis, ultrafiltration and hemodiafiltration.

The systems described herein can include a control system with flow rate control mechanisms for a patient to control the pumps pursuant to the patient-desired time-period of a treatment session. Nocturnal dialysis allows a patient to sleep while performing a longer treatment session such as 6 to 9 or more hours in contrast to typical daytime dialysis, which can typically last 3 to 5 hours per session. Longer treatment sessions have been shown to provide certain advantages to a patient, such as freeing time during busy daily schedules, lower blood phosphate levels, reduced blood pressure, desirable weight gain and improved appetite, and improved sense of well-being. Some patients may nonetheless need to select a shorter treatment period.

The systems described herein can incorporate a control mechanism that is configured to enable a patient to select shorter or longer treatment periods for complete hemodialysis, ultrafiltration or hemodiafiltration of a patient's blood. In this regard, the user provides the control system with a maximum time span over which a dialysis treatment should be completed. The control system is configured to automatically control the dialysate flow rate through the system to achieve the maximum use of the available dialysate within the time span identified by the patient. The control system can automatically adjust the relative speed of one or more pumps of the system to achieve a flow rate such that all dialysate (or an appropriate volume of dialysate) is used during the identified span of time in which the treatment should be completed. That is, the control system can select and implement a higher flow rate of dialysate through the dialysis system to achieve a higher rate of dialysis so as to complete the dialysis treatment within a shorter time span. Likewise, the control system can select and implement a lower flow rate of dialysate through the dialysis system to achieve a lower rate of dialysis so as to complete the dialysis treatment within a relatively shorter time span, as identified by the user. In an embodiment, the control system can select and implement a variety of flow rates during the course of the treatment, for example commencing at a higher flow rate when the patient's blood is the most in need of dialysis, and then reducing the flow rate to one or more lower flow levels as the treatment continues, to ensure both that all of the dialysate is used during the treatment and that the remaining dialysate is sufficient for the planned duration of the treatment.

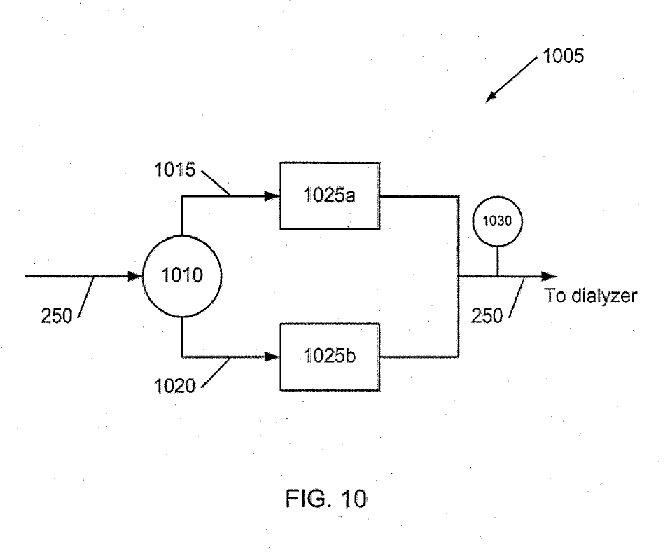

As shown in FIG. 10, the foregoing flow rate control can be achieved using a flow path configuration 1005, which is located along the dialysate inlet pathway 250 upstream of the dialyzer 15. The flow path configuration 1005 includes a valve 1010 that can be toggled to selectively direct the dialyzer through either a first pathway 1015 or a second pathway 1020 as the dialysate flows towards the dialyzer 15. Each of the first and second pathways includes a corresponding flow restrictor 1025 including a first flow restrictor 1025a (located on first pathway 1015) and a second flow restrictor 1025b (located on second pathway 1020). The flow restrictors 1025 are each configured to achieve a predetermined flow rate of dialysate as it flows through the dialysate inlet pathway 250 toward the dialyzer. For example, the first flow restrictor 1025a may achieve a flow rate of 100 ml/min while the second flow restrictor 1025b achieves a flow rate different than that of the first flow restrictor 1025a, such as 300 ml/min. The aforementioned flow rates are examples and other flow rates are within the scope of this disclosure. Also within the scope of this disclosure are systems with more than three or more flow pathways corresponding to three or more flow rates, or a single pathway with an element, such as a pump or variable flow restrictor, than can modify the flow rate in response to the control system and desired control regimen.

The control mechanism can control the valve 1010 to direct the dialysate to flow along a flow pathway with a flow rate that is most likely to achieve dialysis treatment within the time allotted by the patient. For example, if the patient specified a relatively short amount of time for the dialysis treatment, the control system selects the flow restrictor pathway that has a higher flow rate, such as the first pathway 1015. If the patient specified a relatively longer amount of time for the dialysis treatment, the control system selects the flow restrictor pathway that has a lower flow rate, such as the second pathway 1020, as the lower flow rate permits completion of the dialysis treatment over a longer time span. It should be appreciated that the flow path configuration 1005 could include multiple flow pathways each associated with a different flow rate that would complete dialysis within differing time spans. In another embodiment, the control system is configured to set the flow rate of each flow pathway in real time so as to adjust the flow rate on the fly to achieve dialysis within the patient-allotted time span.

With reference still to FIG. 10, a flow rate sensor 1030 can be positioned downstream of the flow restrictors 1025 to sense the actual rate of flow through the dialysate inlet pathway 250. The flow rate sensor 1030 may be in a feedback relationship with the valve 250 so that the flow can be alternated between the flow pathways 1015 and 1020 to thereby adjust the flow rate on the fly depending on how much time is left within the allotted time for completion of treatment. A feed pressure measurement can be taken upstream of the control valves to assess whether or not the correct flow restrictor (and associated flow rate) is selected for the desired treatment time span.

In some embodiments, the patient can select between a daytime setting and a nighttime setting wherein the daytime setting is typically associated with a predetermined shorter treatment time span than the nighttime setting. Or, the patient can select between predetermined spans of time for a treatment session. For example, the control mechanism can be adjusted to an overall treatment session of at least about 2, 3, 4, 5, 6, 7, 8, 9, 10 hours or longer treatment session. The control mechanism can automatically adjust one or more of the pumps to various flow rates, such as for example to provide a flow rate of at least about 10 ml/min, 20 ml/min, 30 ml/min, 40 ml/min, 50 ml/min, 60 ml/min, 70 ml/min, 80 ml/min, 90 ml/min, 100 ml/min, 110 ml/min, 120 ml/min, 130 ml/min, 140 ml/min, 150 ml/min, 200 ml/min, 250 ml/min, 300 ml/min, 350 ml/min, 400 ml/min, 450 ml/min, 500 ml/min, 550 ml/min, 600 ml/min, 700 ml/min, 800 ml/min, 900 ml/min, 1000 ml/min, or greater ml/min. In some implementations, a treatment sessions can be approximately 3 hours long and performed at speeds at least about 300 ml/min. In other implementations, a treatment session can be approximately 8 hours long and performed at speeds at least about 100 ml/min. It should be appreciated that any combination of treatment session length and flow rates can be selected to provide for a customized treatment session.

The patient can adjust the control mechanisms on a user interface such as using a dial, keypad, button, touch-screen or other type of input device configured to enter overall treatment time in minutes and/or hours. In some embodiments, the patient can adjust the flow rate of one or more pumps and the user interface automatically indicates to the patient the length of treatment session for the selected flow rate.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A medical dialysis system, comprising:
a filtration system configured to filter a water stream;
a water purification system configured to purify the water stream in a non-batch process;
a mixing system, comprising:
a supply of one or more dialysate components;
a first conductivity sensor positioned within a fluid pathway through which the water stream flows; and
a control mechanism comprising a pump configured to control an amount of the one or more dialysate components added to the water stream from the supply;
wherein the mixing system is configured to produce a stream of dialysate from mixing the one or more dialysate components with the water stream in a non-batch process;
a dialyzer system, comprising:
a dialyzer configured to fluidly couple to the stream of dialysate and a blood stream and comprising a membrane separating the stream of dialysate from the blood stream facilitating dialysis of the blood stream;
a plurality of pumps capable of pumping the stream of dialysate across the dialyzer; and
a second conductivity sensor positioned downstream of the dialyzer within a fluid pathway through which the water stream flows.

2. The system of claim 1, further comprising a microprocessor configured to calculate a difference between a first electrical measurement by the first conductivity sensor and a second electrical measurement by the second conductivity sensor to determine a transfer rate of electrolytes between the stream of dialysate and the blood stream within the dialyzer.

3. The system of claim 2, wherein the control mechanism of the mixing system adjusts in real-time the amount of the one or more dialysate components mixed into the water stream based on the calculated difference until the transfer rate of electrolytes falls below a predetermined threshold.

4. The system of claim 2, wherein the transfer rate of electrolytes falls below a predetermined threshold as the difference between the first electrical measurement and the second electrical measurement approaches zero.

5. The system of claim 1, wherein an electrolyte level of the blood stream remains unchanged following a treatment with the dialysis system.

6. The system of claim 1, wherein the mixing system further comprises a pressure transducer positioned downstream of the supply and upstream of the control mechanism, wherein the pressure transducer measures fluid pressure from the supply and indicates whether the supply contains sufficient components to continue a dialysis treatment based on the measured fluid pressure.

7. The system of claim 6, wherein the control mechanism monitors the measured fluid pressure from the pressure transducer and compares the measured fluid pressure to a threshold value to assess a status of the supply.

8. The system of claim 7, wherein the control system provides an alert regarding the status of the supply.

9. The system of claim 8, wherein the alert indicates a degree of sufficiency or insufficiency of the supply.

10. A method for matching dialysate electrolyte levels to a patient's blood electrolyte levels during a dialysis treatment, comprising:
purifying a water stream in a non-batch process using a water purification system;
producing a dialysate stream by mixing one or more dialysate components with the water stream in a non-batch process;
measuring electrical conductivity of the dialysate stream using a first conductivity sensor positioned within a fluid pathway upstream of a dialyzer before the dialysate stream contacts a blood stream, the dialysate stream having a first electrolyte concentration;
measuring electrical conductivity of the dialysate stream using a second conductivity sensor positioned within a fluid pathway downstream of the dialyzer after the dialysate stream contacts the blood stream, the dialysate stream having a second electrolyte concentration;
calculating a difference between the first electrical conductivity measurement and the second electrical measurement to determine a transfer rate of electrolytes between the dialysate stream and the blood stream within the dialyzer at a first flow rate; and
adjusting in real-time the first electrolyte concentration using a control mechanism comprising a pump configured to control an amount of the one or more dialysate components mixed with the water stream until the transfer rate of electrolytes falls below a predetermined threshold.

11. The method of claim 10, wherein the transfer rate of electrolytes falls below a predetermined threshold as the difference between the first electrolyte concentration and the second electrolyte concentration approaches zero.

12. The method of claim 10, wherein the dialysate stream contacts the blood stream within the dialyzer across a semi-permeable membrane.

13. The method of claim 10, wherein the blood electrolyte levels remain unchanged following the dialysis treatment.
